(19) United States
(12) Patent Application Publication
(10) Pub. No.: US 2025/0295892 A1
(43) Pub. Date: Sep. 25, 2025

(54) GUIDE WIRE

(71) Applicant: TERUMO KABUSHIKI KAISHA, Tokyo (JP)

(72) Inventors: Gen OMURA, Fujinomiya (JP); Tsutomu SUGIKI, Fujinomiya (JP)

(73) Assignee: TERUMO KABUSHIKI KAISHA, Tokyo (JP)

(21) Appl. No.: 19/229,743

(22) Filed: Jun. 5, 2025

Related U.S. Application Data
(63) Continuation of application No. PCT/JP2023/039701, filed on Nov. 2, 2023.

(30) Foreign Application Priority Data
Dec. 12, 2022 (JP) 2022-198103

(51) Int. Cl. A61M 25/09

(52) U.S. Cl. CPC A61M 25/09; A61M 2025/09058; A61M 2025/09133; A61M 2025/09175; A61M 2205/0238

(57) ABSTRACT

Provided is a guide wire including a wire extending in a longitudinal direction of the guide wire, in which the wire has a groove portion including helical grooves formed on an outer circumference of the wire along a helical path, each of the helical grooves has one or more bridge portions per circumference, and the bridge portion is a portion where a groove is not continuous or a groove depth is ½ or less of a maximum depth per circumference.

### CROSS-REFERENCES TO RELATED APPLICATIONS

This application is a continuation of International Patent Application No. PCT/JP2023/039701 filed on Nov. 2, 2023, which claims priority to Japanese Patent Application No. 2022-198103 filed on Dec. 12, 2022, the entire content of both of which is incorporated herein by reference.

### TECHNOLOGICAL FIELD

The present disclosure generally relates to a guide wire.

### BACKGROUND DISCUSSION

A guide wire inserted into a body lumen such as a blood vessel to assist in the insertion of a medical device such as a catheter or a stent is known (see, for example, JP 2004-230142 A and WO 2021/117657 A).

### SUMMARY

It is difficult for a conventional guide wire to achieve both flexibility and torque transmission.

An object of the present disclosure is to provide a guide wire that easily achieves both flexibility and torque transmission. Aspects of the present disclosure are as follows.
[1] A guide wire including a wire extending in a longitudinal direction of the guide wire, in which the wire has a groove portion including helical grooves formed on an outer circumference of the wire along a helical path, each of the helical grooves has one or more bridge portions per circumference, and the bridge portion is a portion where a groove is not continuous or a groove depth is ½ or less of a maximum depth per circumference.
[2] The guide wire according to [1], in which the bridge portions are disposed at intervals ranging from 150° to 170° or 190° to 210°.
[3] A guide wire including a wire extending in a longitudinal direction of the guide wire, in which the wire has a groove portion including a plurality of circumferential grooves arranged in an axial direction of the wire and extending on an outer circumference of the wire along an annular path, each of the circumferential grooves has one or more bridge portions per circumference, and the bridge portion is a portion where a groove is not continuous or a groove depth is ½ or less of a maximum depth per circumference.
[4] The guide wire according to [3], in which each of the circumferential grooves has two bridge portions facing each other in a radial direction of the wire.
[5] The guide wire according to [4], in which the bridge portions are disposed so as to be shifted by 75° to 85° or 95° to 105° per circumferential groove toward a distal end of the wire.
[6] The guide wire according to any one of [1] to [5], in which the groove portion has a portion where a ratio of a groove width with respect to one groove pitch increases toward a distal end of the wire to increase flexibility.
[7] The guide wire according to [6], in which in the groove portion, the groove width of the portion is constant.
[8] The guide wire according to any one of [1] to [7], in which the groove portion has a portion where a groove becomes deeper toward a distal end of the wire to be flexible.
[9] The guide wire according to any one of [1] to [8], in which the groove portion has a portion where the wire becomes tapered toward a distal end of the wire to be flexible.
[10] The guide wire according to any one of [1] to [9], in which the groove portion has a portion where a circumferential length of the bridge portion becomes shorter toward a distal end of the wire to be flexible.
[11] The guide wire according to any one of [1] to [10], in which a coil or a tube in which slits are formed is provided at a distal portion of the guide wire.
[12] The guide wire according to [11], in which the groove portion is located on a proximal side of at least the distal portion of the guide wire.
[13] The guide wire according to any one of [1] to [10], in which at least a resin coating layer mainly made of a synthetic resin and a hydrophilic outermost layer are provided at a distal portion of the guide wire.

The guide wire disclosed here is able to rather easily achieve both flexibility and torque transmission.

According to another aspect, a guide wire comprises: an elongated solid wire having a longitudinal extent extending from a distal end of the elongated solid wire to a proximal end of the elongated solid wire, with the elongated solid wire having an outer peripheral surface, and wherein the elongated solid wire has a groove portion that extends radially inwardly from the outer peripheral surface of the elongated solid wire so that the groove portion opens to the outer peripheral surface of the elongated solid wire. An outer layer is positioned in covering relation to the outer peripheral surface of the elongated solid wire, inclusive of the groove portion of the elongated solid wire. The groove portion includes: i) helical grooves each extending in a helical manner around the elongated solid wire and extending along at least a portion of the longitudinal extent of the elongated solid wire, the helical grooves having a maximum depth, each of the helical grooves having a bridge portion per each 360° circumferential extent, the bridge portion of each helical groove being a portion of the elongated solid wire in which: i) the helical groove is not continuous; or ii) a depth of the helical groove is ½ or less of the maximum depth per each 360° circumferential extent; or ii) a plurality of circumferential grooves each extending circumferentially in an annular manner around the elongated solid wire and extending along at least a portion of the longitudinal extent of the elongated solid wire so that circumferential grooves positioned axially adjacent each other in an axial direction of the elongated solid wire are axially spaced apart from one another, the circumferential grooves having a maximum depth, each circumferential groove having a bridge portion per each 360° circumferential extent, the bridge portion of each circumferential groove is a portion of the elongated solid wire in which: i) the circumferential groove is not continuous; or ii) a depth of the circumferential groove is ½ or less of the maximum depth per each 360° circumferential extent.

### BRIEF DESCRIPTION OF THE DRAWINGS

FIG. 1 is a longitudinal cross-sectional view illustrating a guide wire according to a first embodiment.

FIG. 2A is a partially enlarged view of a wire illustrated in FIG. 1.

FIG. 2B is a side view of the wire illustrated in FIG. 2A.

FIG. 2C is a partially enlarged view illustrating a modification example of the wire illustrated in FIG. 2A.

FIG. 2D is a partially enlarged view illustrating a modification example of the wire illustrated in FIG. 2C.

FIG. 3A is a cross-sectional perspective view illustrating a modification example of the wire illustrated in FIG. 1.

FIG. 3B is a cross-sectional perspective view illustrating a modification example of the wire illustrated in FIG. 3A.

FIG. 4A is a side view illustrating a modification example of the wire illustrated in FIG. 2B.

FIG. 4B is a side view illustrating a modification example of the wire illustrated in FIG. 4A.

FIG. 5 is a cross-sectional perspective view illustrating a modification example of the wire illustrated in FIG. 4A.

FIG. 6 is a longitudinal sectional view illustrating a guide wire according to a second embodiment.

FIG. 7 is a longitudinal sectional view illustrating a guide wire according to a third embodiment.

FIG. 8 is a partially enlarged view of FIG. 7.

FIG. 9 is a longitudinal sectional view illustrating a guide wire according to a fourth embodiment.

FIG. 10 is a longitudinal sectional view illustrating a guide wire according to a fifth embodiment.

FIG. 11 is a longitudinal sectional view illustrating a guide wire according to a sixth embodiment.

FIG. 12 is a longitudinal sectional view illustrating a guide wire according to a seventh embodiment.

FIG. 13 is a longitudinal sectional view illustrating a guide wire according to an eighth embodiment.

### DETAILED DESCRIPTION

Embodiments of a guide wire, representing examples of the new guide wire disclosed here, will be described in detail below with reference to the accompanying drawings.

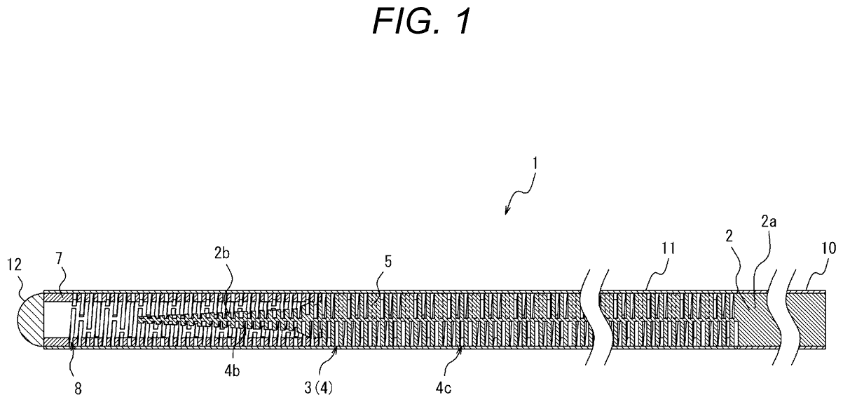

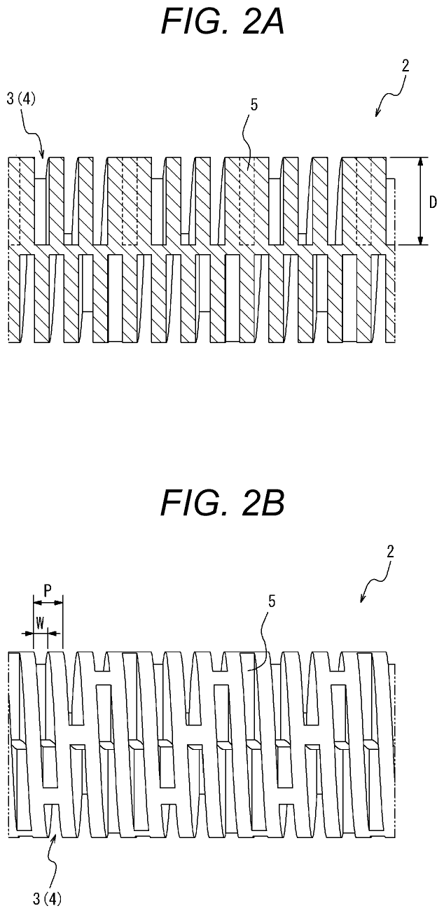

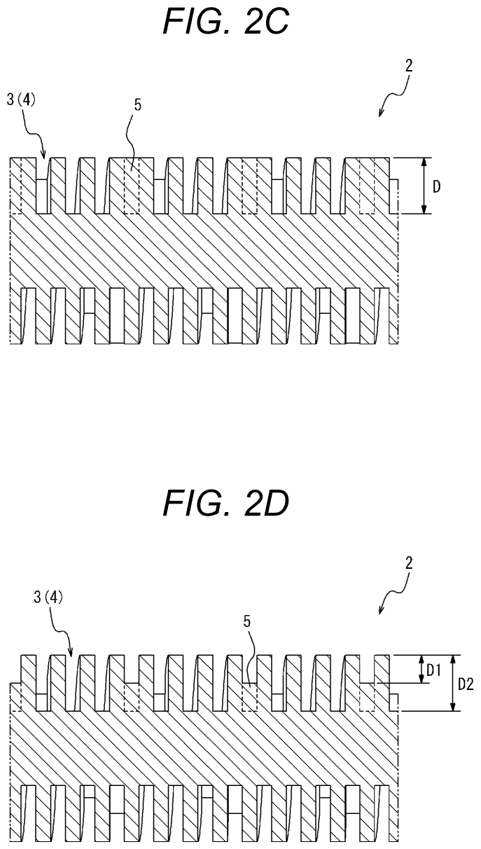

As illustrated in FIGS. 1 to 2B, a guide wire 1 according to a first embodiment includes a wire 2 (elongated solid wire) extending in a longitudinal direction of the guide wire 1, the wire 2 includes a groove portion 4 including helical grooves 3 formed on an outer circumference of the wire 2 along a helical path, each of the helical groove 3 has one or more bridge portions 5 per circumference (per each 360° circumferential extent), and the bridge portion 5 is a portion where the groove is not continuous.

In such a configuration, the wire 2 has the helical grooves 3, and thus the flexibility of the guide wire 1 (the performance that the guide wire 1 can be flexibly bent along the shape of a blood vessel or the like) can be enhanced, and the helical grooves 3 have the sufficient number of bridge portions 5, and thus the torque transmission performance (the performance capable of transmitting the rotation operation in a twisting direction, that is, in a circumferential direction at the proximal portion of the guide wire 1 to the distal portion of the guide wire 1) of the guide wire 1 can be favorably maintained. Therefore, both the flexibility and the torque transmission performance can be easily achieved.

The groove depth D of the helical groove 3 can be appropriately set according to the application, material, and the like of the guide wire 1. As an example, a modification example in which the groove depth D of the helical grooves 3 is reduced is illustrated in FIG. 2C. That is, FIG. 2C shows a guide wire with grooves 3 having a reduced or smaller depth compared to the grooves 3 in the FIG. 2A embodiment.

The bridge portion 5 may have a configuration in which the groove depth D1 of the bridge portion 5 is ½ or less of the maximum depth D2 of the helical groove 3 per circumference, instead of the configuration in which the bridge portion 5 is a portion where the groove is not continuous. Even in such a configuration, the above-described effect can be achieved to a certain extent. FIG. 2D illustrates a modification example in which the groove depth D1 of the bridge portion 5 is ½ of the maximum depth D2 of the helical groove 3 per circumference.

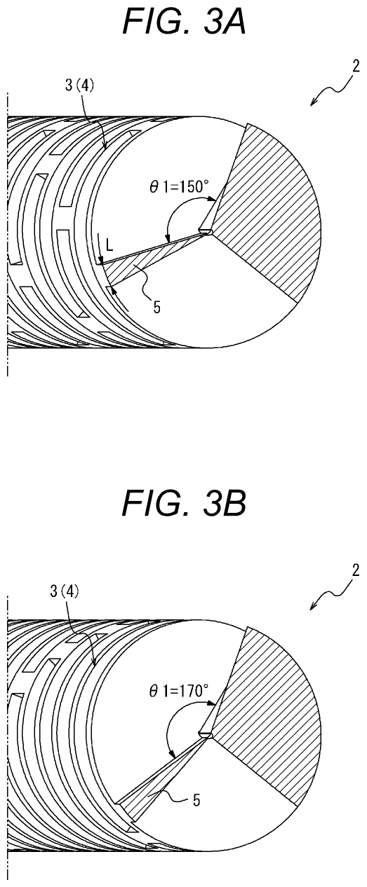

The bridge portions 5 are preferably disposed at intervals ranging from 150° to 170° or 190° to 210°, for example, as in the modification examples illustrated in FIGS. 3A to 3B. That is, an angle θ1 at which two bridge portions 5 adjacent to each other along the helical groove 3 are shifted from each other in the circumferential direction preferably ranges from 150° to 170° or 190° to 210°. FIG. 3A is an example of 150°, and FIG. 3B is an example of 170°. In such a configuration, the helical grooves 3 have the bridge portions 5 at intervals approximately half a circumference, that is, slightly shifted from 180° (the bridge portions 5 in each helical groove 3 are thus circumferentially shifted relative to all bridge portions 5 in the axially adjacent helical grooves 3), and thus it is possible to achieve a structure in which the helical grooves 3 have the bridge portions 5 circumferentially distributed at appropriate intervals. Therefore, it is possible to easily achieve flexibility in all directions and good torque transmission.

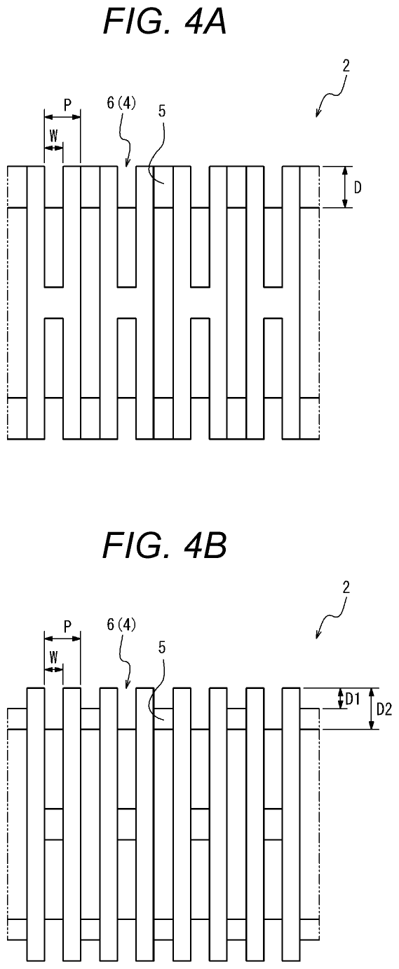

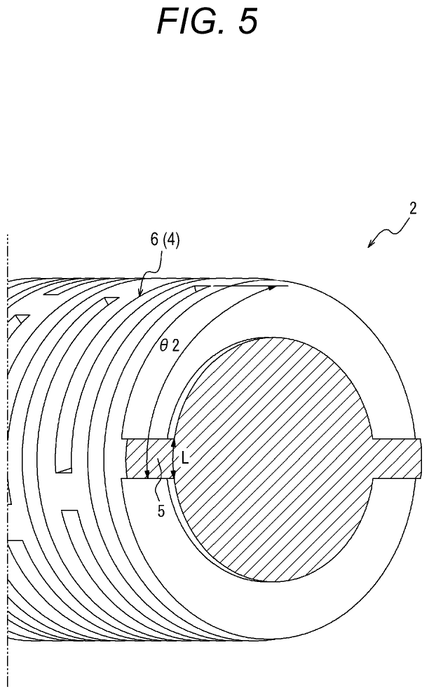

Instead of the configuration including the helical grooves 3, the groove portion 4 may include a plurality of circumferential grooves 6 as illustrated in FIG. 4A. In this case, a plurality of the circumferential grooves 6 is arranged in an axial direction of the wire 2 and each of the circumferential grooves 6 extends on the outer circumference of the wire 2 along an annular path, each circumferential groove 6 has one or more bridge portions 5 per circumference, and the bridge portion 5 is a portion where the groove is not continuous.

In such a configuration, the wire 2 has a plurality of the circumferential grooves 6, and thus the flexibility of the guide wire 1 can be enhanced, and a plurality of the circumferential grooves 6 has the sufficient number of bridge portions 5, and thus the good torque transmission of the guide wire 1 can be maintained. Therefore, both the flexibility and the torque transmission can be easily achieved.

The groove depth D of each of a plurality of the circumferential grooves 6 can be appropriately set according to the application, material, and the like of the guide wire 1.

As illustrated in FIG. 4B, the bridge portion 5 may have a configuration in which the groove depth D1 of the bridge portion 5 is ½ or less of the maximum depth D2 of the circumferential groove 6 per circumference, instead of the configuration in which the bridge portion 5 is a portion where the groove is not continuous. Even in such a configuration, the above-described effect can be achieved to a certain extent.

As illustrated in FIGS. 4A and 4B, and FIG. 5, each of the circumferential grooves 6 may have a configuration in which the bridge portion 5 is provided at two locations facing each other in a radial direction of the wire 2. In such a configuration, it is possible to achieve a structure in which the groove portion 4 has the bridge portions 5 at appropriate intervals. Therefore, it is possible to easily achieve good torque transmission.

As illustrated in FIG. 5, the bridge portion 5 may be disposed so as to be shifted by 75° to 85° or 95° to 105° per circumferential groove 6 (that is, an angle θ2 at which the bridge portion 5 is shifted per circumferential groove 6 ranges from 75° to 85° or 95° to 105°) toward the distal end of the wire 2. Thus, as shown in FIG. 5, there are two bridge portions 5 spanning each circumferential groove 6, and the two bridge portions 5 are offset or shifted (circumferentially offset/shifted) by 75° to 85° or 95° to 105° relative to each other. In such a configuration, the groove portion 4 has two bridge portions 5 facing each other at positions slightly shifted from right angle position or 90° per circumferential groove 6, and thus it is possible to achieve a structure in which the groove portion 4 has the bridge portions 5 circumferentially distributed at appropriate intervals. Therefore, it is possible to easily achieve flexibility in all directions and good torque transmission.

As illustrated in FIGS. 4A and 4B, and FIG. 5, each of the circumferential grooves 6 may extend along the circumferential direction of the wire 2 (that is, the annular path extends along the circumferential direction of the wire 2). In such a configuration, both flexibility and torque transmission can be more reliably achieved.

The groove portion 4 may have a portion where the ratio of the groove width W to one groove pitch P increases toward the distal end of the wire 2 to increase flexibility. FIG. 2B illustrates a groove pitch P and a groove width W in a case where the groove portion 4 includes the helical grooves 3, and FIGS. 4A and 4B illustrate a groove pitch P and a groove width W in a case where the groove portion 4 includes a plurality of circumferential grooves 6. In such a configuration, the flexibility of the guide wire 1 can be optimized with a simple structure.

The groove portion 4 may have a portion where the ratio of the groove width W to one groove pitch P increases toward the distal end of the wire 2 to increase flexibility and the groove width W may be constant. In such a configuration, for example, the wire 2 can be easily manufactured using a laser.

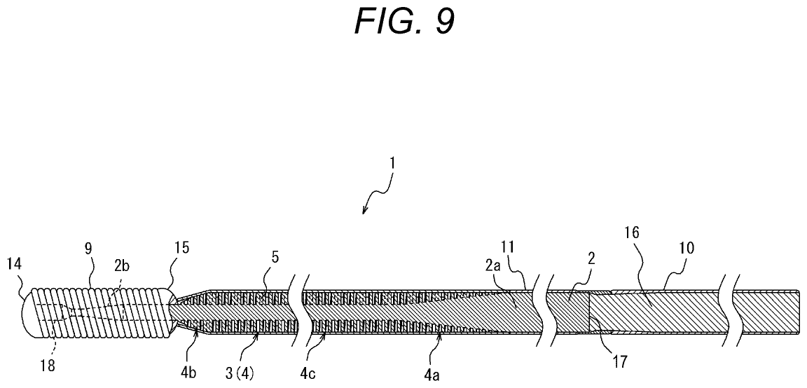

The groove portion 4 may have a portion (also referred to as a deep groove portion 4a) where the groove becomes deeper and so that the wire 2 becomes more flexible toward the distal end of the wire 2. Examples of the deep groove portion 4a are illustrated in FIGS. 9 to 13. FIGS. 9 to 13 illustrate examples in which the groove portion 4 includes the helical grooves 3, but the same applies to a case where the groove portion 4 includes a plurality of circumferential grooves 6. In such a configuration, the flexibility of the guide wire 1 can be optimized with a simple structure.

The groove portion 4 may have a portion (also referred to as a tapered wire portion 4b) where the wire 2 tapers in a narrowing manner and becomes flexible toward the distal end of the wire 2. Examples of the tapered wire portion 4b are illustrated in FIGS. 1, 6, 9, 10, and 13. FIGS. 1, 6, 9, 10, and 13 illustrate examples in which the groove portion 4 includes the helical grooves 3, but the same applies to a case where the groove portion 4 includes a plurality of circumferential grooves 6. In such a configuration, the flexibility of the guide wire 1 can be optimized with a simple structure.

The groove portion 4 may have a portion where the circumferential length L of the bridge portion 5 becomes shorter toward the distal end of the wire 2 and becomes flexible. FIG. 3A illustrates the circumferential length L of the bridge portion 5 in a case where the groove portion 4 includes the helical grooves 3, and FIG. 5 illustrates the circumferential length L of the bridge portion 5 in a case where the groove portion 4 includes a plurality of the circumferential grooves 6. In such a configuration, the flexibility of the guide wire 1 can be optimized with a simple structure.

As in the examples illustrated in FIGS. 1, 6, and 7, a tube or tubular body 7 in which slits 8 are formed may be provided at the distal portion of the guide wire 1. FIGS. 1, 6, and 7 illustrate examples in which the groove portion 4 includes the helical grooves 3, but the same applies to a case where the groove portion 4 includes a plurality of circumferential grooves 6. In such a configuration, for example, radiopacity can be imparted to the distal portion of the guide wire 1 by the tube 7 made of a material with high X-ray contrast, and the flexibility of the distal portion of the guide wire 1 can be secured by the slits 8 formed in the tube 7.

In the examples illustrated in FIGS. 1, 6, and 7, each of the slits 8 of the tube 7 has a helical shape, and has one or more non-continuous portions per circumference (per circumferential) (360° extent). However, the slits 8 are not limited to such a configuration. For example, the slits 8 may include a plurality of annular slits arranged in the axial direction of the pipe 7, and each of the annular slits may have one or more non-continuous portions per circumference.

As in the example illustrated in FIG. 9, a coil 9 may be provided at the distal portion of the guide wire 1. FIG. 9 illustrates an example in which the groove portion 4 includes the helical grooves 3, but the same applies to a case where the groove portion 4 includes a plurality of circumferential grooves 6. In such a configuration, for example, high X-ray contrast can be imparted to the distal portion of the guide wire 1 by the coil 9 made of a material with radiopacity, and the flexibility of the distal portion of the guide wire 1 can be secured by the flexibility of the coil 9. On the other hand, the groove portion 4 provided on the proximal side of the coil 9 can achieve both flexibility and torque transmission.

From the viewpoint of easily achieving both the flexibility and the torque transmission, it is preferable that the groove portion 4 is located on the proximal side of at least the distal portion of the guide wire 1 as illustrated in FIGS. 1, 6, 7, and 9 to 13. FIGS. 1, 6, 7, and 9 to 13 illustrate examples in which the groove portion 4 includes the helical grooves 3, but the same applies to a case where the groove portion 4 includes a plurality of circumferential grooves 6.

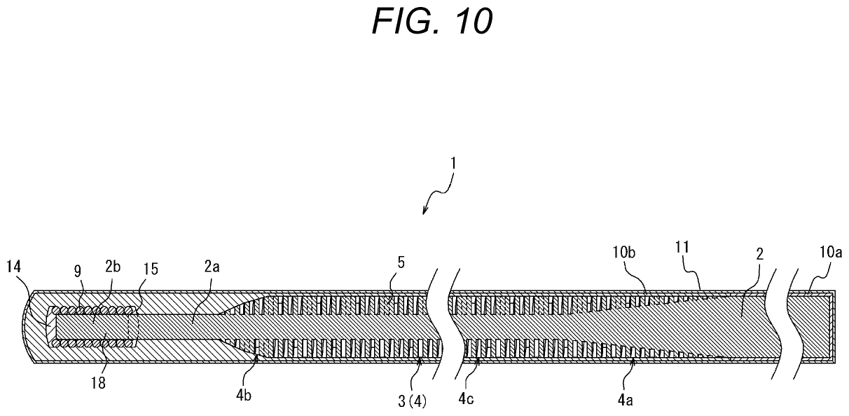

As illustrated in FIGS. 10 to 13, a configuration may be adopted in which a resin coating layer 10 mainly made of a synthetic resin and a hydrophilic outermost layer 11 are provided at least at the distal portion of the guide wire 1. FIGS. 10 to 13 illustrate examples in which the groove portion 4 includes the helical grooves 3, but the same applies to a case where the groove portion 4 includes a plurality of circumferential grooves 6. In such a configuration, both the flexibility and the torque transmission can be more reliably achieved.

The guide wire 1 of the first embodiment illustrated in FIG. 1 includes a distal member 12, a tube 7, and a wire 2. The distal end of the guide wire 1 is constituted by the distal member 12, and the distal member 12 is fixed to the distal portion of the tube 7. The wire 2 includes a wire body 2a extending from the proximal portion of the guide wire 1 to the proximal side end portion (proximal portion) of the tube 7, and an insertion portion 2b inserted into or positioned in the tube 7 from the distal end of the wire body 2a, separated from the inner circumferential surface of the tube 7, and extending toward the distal portion of the tube 7. The groove portion 4 is provided at a portion ranging from a portion between the proximal portion and the distal portion of the wire body 2a to the distal portion and the insertion portion 2b. The distal end of the insertion portion 2b is provided on the proximal side of the distal member 12. The insertion portion 2b has a tapered shape in which the thickness of the wire 2 gradually decreases from the proximal portion toward the distal side.

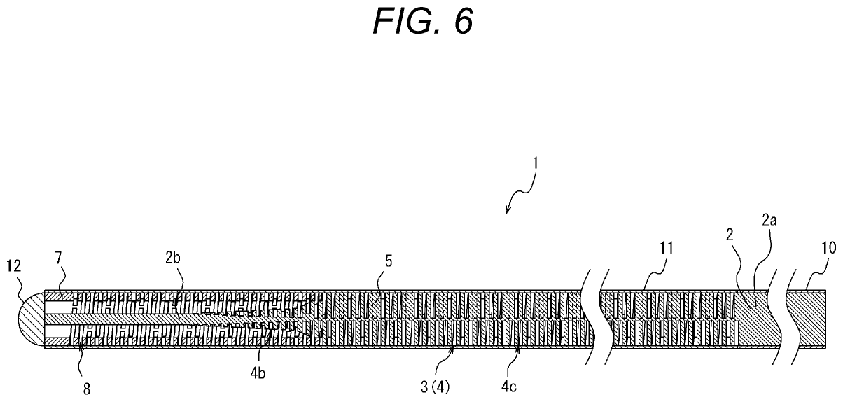

In the second embodiment illustrated by way of example in FIG. 6, the wire 2 may be configured such that the distal end of the insertion portion 2b is provided at a position in contact with the distal member 12 and is fixed to the distal member 12.

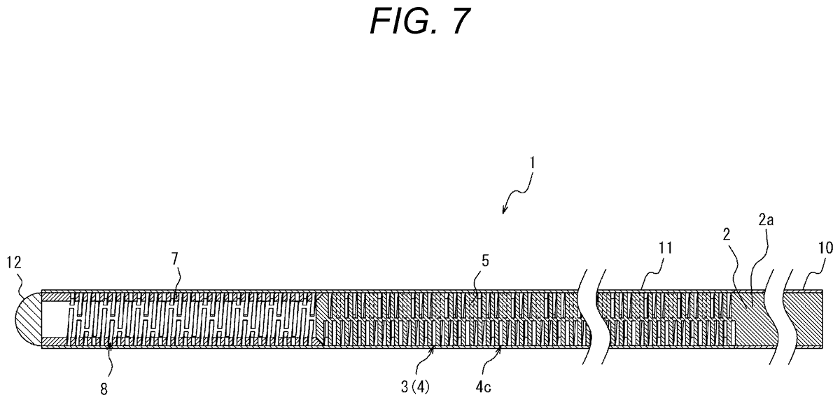

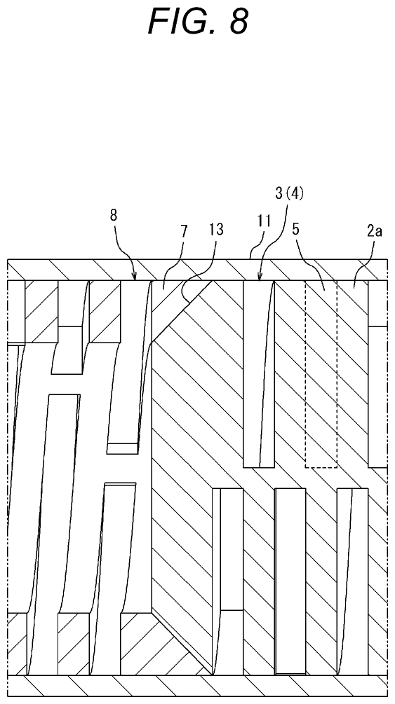

The wire 2 may not include the insertion portion 2b as in the third embodiment illustrated in FIG. 7. In this case, as shown in FIG. 8, a joint surface 13 between the proximal portion of the tube 7 and the distal portion of the wire body 2a is preferably configured as an inclined surface inclined with respect to a surface perpendicular to the longitudinal direction of the wire 2 from the viewpoint of enhancing the joint strength.

The guide wire 1 according to the first to third embodiments includes the resin coating layer 10 on the outer surface of the portion of the wire body 2a excluding the groove portion 4, and includes the hydrophilic outermost layer 11 on the outer surfaces of the groove portion 4 and the tube 7 in the wire body 2a.

In the first to third embodiments, the wire 2 can be formed of, for example, a nickel-titanium alloy (Ni—Ti alloy). The tube 7 can be formed of, for example, tungsten. The resin coating layer 10 can be formed of, for example, polytetrafluoroethylene (PTFE) or the like. The tube 7 and the wire 2 can be joined by, for example, adhesion, welding, or the like.

A guide wire 1 of a fourth embodiment illustrated in FIG. 9 includes a coil 9, a coil distal member 14, a coil proximal member 15, a wire 2, and a proximal side core member 16. The distal end of the coil 9 is fixed to the wire 2 via the coil distal member 14, and the proximal end of the coil 9 is fixed to the wire 2 via the coil proximal member 15. The wire 2 includes a wire body 2a extending from a joint portion 17 with the proximal side core member 16 to the coil proximal member 15, and an insertion portion 2b inserted into the coil 9 from the distal end of the wire body 2a, separated from the inner circumferential surface of the coil 9, and extending toward the distal portion of the coil 9. The groove portion 4 is provided from a portion between the proximal portion (joint portion 17) and the distal portion of the wire body 2a toward the distal side.

In the guide wire 1 of the fourth embodiment, the distal end of the insertion portion 2b is fixed to the coil distal member 14.

In the guide wire 1 of the fourth embodiment, the distal portion of the insertion portion 2b includes a plate-like reshapable portion 18. The reshapable portion 18 can be formed by, for example, annealing, work hardening, or the like.

The guide wire 1 of the fourth embodiment includes the deep groove portion 4a as previously described above (the thickness of the wire 2 is constant), a constant portion 4c in which the thickness of the wire 2 and the depth of the groove are constant, and a tapered wire portion 4b as previously described above (the depth of the groove and the thickness of the wire 2 gradually decrease from the proximal portion toward the distal side) in this order from the proximal side toward the distal side.

The constant portion 4c also exists in the first to third embodiments.

The guide wire 1 of the fourth embodiment includes the resin coating layer 10 on the outer surface of the portion of the wire body 2a on the proximal side of the groove portion 4, and includes the hydrophilic outermost layer 11 on the outer surface of the portion of the wire body 2a including the groove portion 4 and the coil 9.

In the fourth embodiment, the wire 2 can be formed of, for example, a nickel-titanium alloy (Ni—Ti alloy). The coil 9 can be formed of, for example, metal having high X-ray contrast such as a platinum-iridium alloy (Pt—Ir alloy), gold (Au), or the like. The proximal side core member 16 can be formed of, for example, stainless steel or the like. The resin coating layer 10 can be formed of, for example, polytetrafluoroethylene (PTFE) or the like.

A guide wire 1 of a fifth embodiment illustrated in FIG. 10 includes a coil 9, a coil distal member 14, a coil proximal member 15, and a wire 2. The distal end of the coil 9 is fixed to the wire 2 via the coil distal member 14, and the proximal end of the coil 9 is fixed to the wire 2 via the coil proximal member 15. The wire 2 includes a wire body 2a extending from the proximal portion to the coil proximal member 15, and an insertion portion 2b inserted into the coil 9 from the distal end of the wire body 2a and extending toward the distal portion of the coil 9. The groove portion 4 is provided from a portion between the proximal portion and the distal portion of the wire body 2a toward the distal side.

In the guide wire 1 of the fifth embodiment, the distal end of the insertion portion 2b is fixed to the coil distal member 14.

In the guide wire 1 of the fifth embodiment, the insertion portion 2b includes a reshapable portion 18. The reshapable portion 18 can be formed by, for example, annealing, work hardening, or the like.

The guide wire 1 of the fifth embodiment includes a deep groove portion 4a (the thickness of the wire 2 is constant), a constant portion 4c in which the thickness of the wire 2 and the depth of the groove are constant, and a tapered wire portion 4b (the depth of the groove becomes shallower toward the distal end and the thickness of the wire 2 gradually decreases from the proximal portion toward the distal side) in this order from the proximal side toward the distal side.

The guide wire 1 of the fifth embodiment includes a first resin coating layer 10a on the outer surface of the portion of the wire body 2a on the proximal side of the groove portion 4, includes a second resin coating layer 10b on the outer surface of the portion of the wire body 2a including the groove portion 4 and the coil 9, and includes a hydrophilic outermost layer 11 on the outer surface sides of the first resin coating layer 10a and the second resin coating layer 10b.

In the fifth embodiment, the wire 2 can be formed of, for example, a nickel-titanium alloy (Ni—Ti alloy). The coil 9 can be formed of, for example, metal having high X-ray contrast such as gold (Au), a platinum-iridium alloy (Pt—Ir alloy), or the like. The first resin coating layer 10a can be formed of, for example, polytetrafluoroethylene (PTFE) or the like. The second resin coating layer 10b can be formed of, for example, polyurethane.

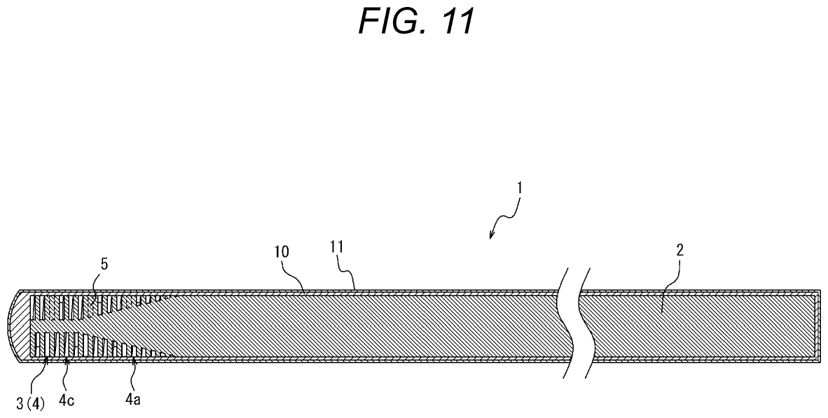

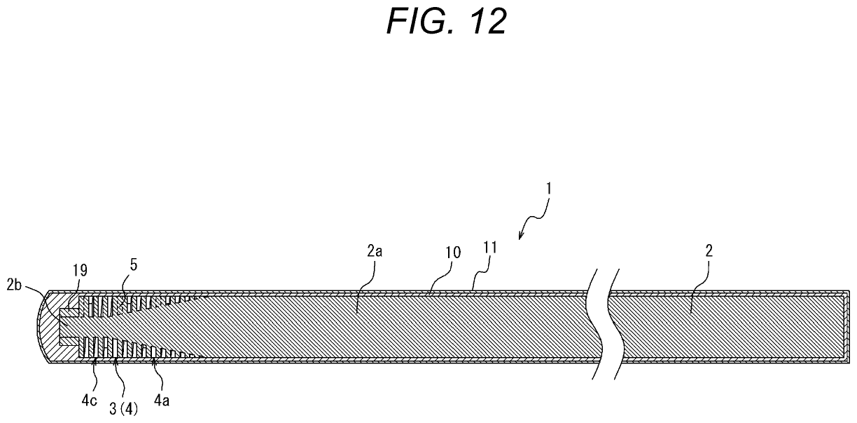

A guide wire 1 of a sixth embodiment illustrated in FIG. 11 includes a wire 2 extending from the proximal portion to the distal portion of the guide wire 1. The groove portion 4 is provided from a portion between the proximal portion and the distal portion of the wire 2 toward the distal side.

The guide wire 1 of the sixth embodiment includes a deep groove portion 4a (the thickness of the wire 2 is constant) and a constant portion 4c in which the thickness of the wire 2 and the depth of the groove are constant in this order from the proximal side toward the distal side.

In the guide wire 1 of the sixth embodiment, the constant portion 4c extends to the distal portion of the guide wire 1.

The guide wire 1 of the sixth embodiment includes a resin coating layer 10 on the outer surface of the wire 2 from the proximal portion to the distal portion, and includes a hydrophilic outermost layer 11 on the outer surface side of the resin coating layer 10.

In the sixth embodiment, the wire 2 can be formed of, for example, a nickel-titanium alloy (Ni-Ti alloy). The resin coating layer 10 can be formed of, for example, polyurethane containing fine tungsten powder.

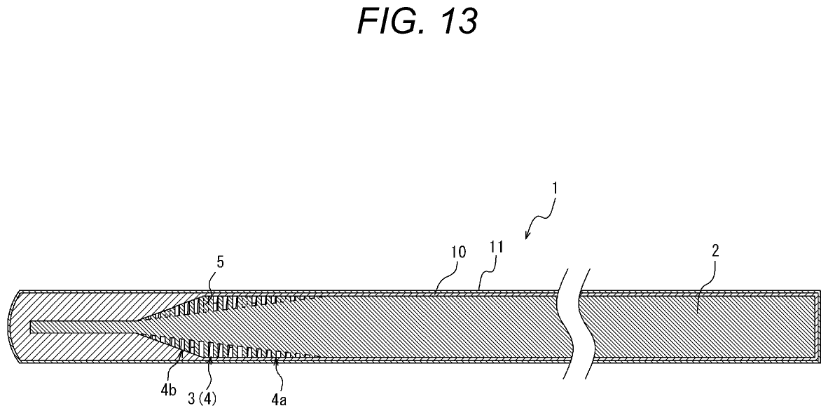

A guide wire 1 of a seventh embodiment illustrated in FIG. 12 includes a

wire 2 extending from the proximal portion to the distal portion of the guide wire 1 and a radiopaque member 19. The wire 2 includes a wire body 2a extending from the proximal portion to the radiopaque member 19, and an insertion portion 2b inserted into or positioned in the annular radiopaque member 19 from the distal end of the wire body 2a and extending toward the distal portion of the radiopaque member 19. The radiopaque member 19 is fixed to the insertion portion 2b. The groove portion 4 is provided from a portion between the proximal portion and the distal portion of the wire 2 toward the distal side.

The guide wire 1 of the seventh embodiment includes a deep groove portion 4a (the thickness of the wire 2 is constant) and a constant portion 4c in which the thickness of the wire 2 and the depth of the groove are constant in this order from the proximal side toward the distal side.

The guide wire 1 of the seventh embodiment includes a resin coating layer 10 on the outer surfaces of the wire 2 and the radiopaque member 19 from the proximal portion to the distal portion, and includes a hydrophilic outermost layer 11 on the outer surface side of the resin coating layer 10.

In the guide wire 1 of the seventh embodiment, the resin coating layer 10 has an increased depth at the distal portion of the guide wire 1 such that the thickness of the guide wire 1 is constant from the proximal portion to the distal portion.

In the seventh embodiment, the wire 2 can be formed of, for example, a nickel-titanium alloy (Ni—Ti alloy). The radiopaque member 19 can be formed of, for example, metal having high X-ray contrast such as gold (Au), a platinum-iridium alloy (Pt—Ir alloy), or the like. The resin coating layer 10 can be formed of, for example, polyurethane containing fine tungsten powder.

A guide wire 1 of an eighth embodiment illustrated in FIG. 13 includes a wire 2 extending from the proximal portion to the distal portion of the guide wire 1. The groove portion 4 is provided from a portion between the proximal portion and the distal portion of the wire 2 toward the distal side. A deep groove portion 4a (the thickness of the wire 2 is constant) and a tapered wire portion 4b are provided in this order from the proximal side toward the distal side.

The guide wire 1 of the eighth embodiment has a portion where the thickness of the wire 2 is constant and the groove portion 4 is not provided on the distal side of the tapered wire portion 4b which is also configured as the deep groove portion 4a.

The guide wire 1 of the eighth embodiment includes a resin coating layer 10 on the outer surface of the wire 2 from the proximal portion to the distal portion, and includes a hydrophilic outermost layer 11 on the outer surface side of the resin coating layer 10.

In the guide wire 1 of the eighth embodiment, the resin coating layer 10 has an increased depth at the distal portion of the guide wire 1 such that the thickness of the guide wire 1 is constant from the proximal portion to the distal portion.

In the eighth embodiment, the wire 2 can be formed of, for example, a nickel-titanium alloy (Ni—Ti alloy). The resin coating layer 10 can be formed of, for example, polyurethane containing fine tungsten powder.

The present disclosure is not limited to the above-described embodiments and may be modified in various ways without departing from the gist of the present disclosure.

Therefore, the guide wire 1 according to the above-described embodiments can be variously modified as long as the guide wire 1 includes the wire 2 extending in the longitudinal direction of the guide wire 1, the wire 2 includes the groove portion 4 including the helical grooves 3 formed on the outer circumference of the wire 2 along the helical path or the groove portion 4 including a plurality of the circumferential grooves 6 arranged in the axial direction of the wire 2 and extending on the outer circumference of the wire 2 along the annular path, the helical groove 3 or each of the circumferential grooves 6 in the groove portion 4 has one or more bridge portions 5 per circumference, and the bridge portion 5 is a portion where the groove is not continuous or the groove depth is ½ or less of the maximum depth per circumference.

The detailed description above describes embodiments of a guide wire representing examples of the new guide wire disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

### REFERENCE SIGNS LIST

1 Guide wire
2 Wire
2a Wire body
2b Insertion portion
3 Helical groove
4 Groove portion
4a Deep groove portion
4b Tapered wire portion
4c Constant portion
5 Bridge portion
6 Circumferential groove
7 Tube
8 Slit
9 Coil
10 Resin coating layer
10a First resin coating layer
10b Second resin coating layer
11 Hydrophilic outermost layer
12 Distal member
13 Joint surface
14 Coil distal member
15 Coil proximal member
16 Proximal side core member
17 Joint portion
18 Reshapable portion
19 Radiopaque member

What is claimed is:

1. A guide wire comprising:
a wire extending in a longitudinal direction of the guide wire, the wire having a groove portion including helical grooves formed on an outer circumference of the wire along a helical path, the helical grooves having a maximum depth, each of the helical grooves having one or more bridge portions per each 360° circumferential extent, the one or more bridge portions of each helical groove being a portion of the wire in which: i) the helical groove is not continuous; or ii) a depth of the helical groove is ½ or less of the maximum depth per each 360° circumferential extent.

2. The guide wire according to claim 1, wherein the one or more bridge portions are disposed at circumferential intervals ranging from 150° to 170° or from 190° to 210°.

3. The guide wire according to claim 1, wherein the one or more bridge portions in each helical groove are circumferentially shifted relative to all the bridge portions in axially adjacent helical grooves.

4. The guide wire according to claim 1, wherein the wire includes a distal portion and a proximal portion, the distal portion of the wire being a tapered portion in which a width of the wire narrows in a direction away from the proximal portion, the tapered portion of the wire and the proximal portion of the wire including the helical grooves having the one or more bridge portions.

5. The guide wire according to claim 4, wherein the distal portion of the wire includes a part having a constant thickness.

6. The guide wire according to claim 1, wherein the helical grooves have a groove width and a groove pitch, each helical groove having a ratio of the groove width to the groove pitch, the ratio of the groove width to the groove pitch of each helical increasing toward a distal end of the wire to increase flexibility.

7. The guide wire according to claim 1, wherein a depth of a part of the groove portion increases toward a distal end of the wire to increase flexibility.

8. A guide wire comprising:
a wire extending in a longitudinal direction of the guide wire,
the wire having a groove portion including a plurality of circumferential grooves arranged in an axial direction of the wire, the circumferential grooves having a maximum depth, each of the circumferential grooves extending on an outer circumference of the wire along an annular path, each of the circumferential grooves having one or more bridge portions per each 360° circumferential extent, the one or more bridge portions of each circumferential groove is a portion of the wire in which: i) the circumferential groove is not continuous; or ii) a depth of the circumferential groove is ½ or less of the maximum depth per each 360° circumferential extent.

9. The guide wire according to claim 8, wherein each of the circumferential grooves has two bridge portions crossing the circumferential groove and facing each other in a radial direction of the wire.

10. The guide wire according to claim 9, wherein the two bridge portions crossing the circumferential groove are circumferentially shifted by 75° to 85° or 95° to 105° per circumferential groove along the wire.

11. The guide wire according to claim 8, wherein the groove portion has a groove width and a groove pitch, a ratio of the groove width to the groove pitch increasing toward a distal end of the wire to increase flexibility.

12. The guide wire according to claim 11, wherein the groove width in at least a part of the groove portion is constant.

13. The guide wire according to claim 8, wherein the groove portion has a groove depth, the groove depth of a portion of the groove becoming deeper toward a distal end of the wire to increase flexibility.

14. The guide wire according to claim 8, wherein a portion of the wire that includes the groove portion is tapered in a narrowing manner toward a distal end of the wire to increase flexibility.

15. The guide wire according to claim 8, wherein the bridge portions each have a circumferential length, the circumferential length of the bridge portions becoming shorter toward a distal end of the wire to increase flexibility.

16. The guide wire according to claim 8, wherein a coil or a tube in which slits are formed is provided at a distal portion of the guide wire.

17. The guide wire according to claim 16, wherein the groove portion is located on a proximal side of at least the distal portion of the guide wire.

18. The guide wire according to claim 8, wherein at least a resin coating layer mainly made of a synthetic resin and a hydrophilic outermost layer are provided at a distal portion of the guide wire.

19. A guide wire comprising:
an elongated solid wire having a longitudinal extent extending from a distal end of the elongated solid wire to a proximal end of the elongated solid wire, the elongated solid wire having an outer peripheral surface;
the elongated solid wire having a groove portion that extends radially inwardly from the outer peripheral surface of the elongated solid wire so that the groove portion opens to the outer peripheral surface of the elongated solid wire;
an outer layer positioned in covering relation to the outer peripheral surface of the elongated solid wire, inclusive of the groove portion of the elongated solid wire; and
the groove portion including:
i) helical grooves each extending in a helical manner around the elongated solid wire and extending along at least a portion of the longitudinal extent of the elongated solid wire, the helical grooves having a maximum depth, each of the helical grooves having a bridge portion per each 360° circumferential extent, the bridge portion of each helical groove being a portion of the elongated solid wire in which: i) the helical groove is not continuous; or ii) a depth of the helical groove is ½ or less of the maximum depth per each 360° circumferential extent; or
ii) a plurality of circumferential grooves each extending circumferentially in an annular manner around the elongated solid wire and extending along at least a portion of the longitudinal extent of the elongated solid wire so that circumferential grooves positioned axially adjacent each other in an axial direction of the elongated solid wire are axially spaced apart from one another, the circumferential grooves having a maximum depth, each circumferential groove having a bridge portion per each 360° circumferential extent, the bridge portion of each circumferential groove is a portion of the elongated solid wire in which: i) the circumferential groove is not continuous; or ii) a depth of the circumferential groove is ½ or less of the maximum depth per each 360° circumferential extent.

20. The guide wire according to claim 19, wherein the bridge portion is one of a pair of bridge portions.
